(19) United States
(12) Patent Application Publication
(10) Pub. No.: US 2026/0198764 A1
(43) Pub. Date: Jul. 16, 2026

(54) VALVE FOR AN ENDOSCOPE

(71) Applicant: Boston Scientific Scimed, Inc., Maple Grove, MN (US)

(72) Inventors: Jasmine Bourgerie, Sherborn, MA (US); Caleb A. Valdes, Lowell, MA (US)

(73) Assignee: Boston Scientific Scimed, Inc., Maple Grove, MN (US)

(21) Appl. No.: 19/447,301

(22) Filed: Jan. 13, 2026

Related U.S. Application Data
(60) Provisional application No. 63/745,046, filed on Jan. 14, 2025.

(51) Int. Cl. A61B 1/015; A61B 1/12; A61M 39/22; F16K 11/07; F16K 31/06

(52) U.S. Cl. CPC A61B 1/015; A61B 1/126; A61M 39/22; F16K 11/0716; F16K 31/0613; A61M 2039/226; A61M 2205/0272

(57) ABSTRACT

Devices, systems, and methods for a valve assembly for a medical device. The valve assembly may include an interface assembly, a valve stem, and a valve body. The interface assembly may include a first portion, a second portion, and a biasing member integrally formed with the first portion and configured to bias the first portion in a proximal direction relative to the second member. The biasing member may be configured to return the first portion to an unactuated position after the first portion is depressed in a distal direction relative to the second portion to an actuated position. The first portion and the biasing member may be entirely or at least partially formed from a unitary single material.

### CROSS-REFERENCE TO RELATED APPLICATIONS

This application claims the benefit of U.S. Provisional Patent Application Serial No. 63/745,046 filed on January 14, 2025, the disclosure of which is incorporated herein by reference.

### TECHNICAL FIELD

This disclosure relates generally to valve assemblies and methods, and particularly to seals for valves of endoscopes and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In an example, an interface assembly for a valve of a medical device may include a first portion, a second portion, and a biasing member that may be integrally formed with the first portion and configured to bias the first portion in a proximal direction relative to a second portion, wherein the biasing member may be configured to return the first portion to an unactuated position after the first portion is advanced in a distal direction relative to the second portion to an actuated position.

Alternatively or additionally to any of the examples above, the first portion, the second portion, and the biasing member may be integrally formed from a monolithic single material.

Alternatively or additionally to any of the examples above, the biasing member and the first portion may be formed from a monolithic single material and the biasing member may be configured to engage the second portion as the first portion adjusts between the unactuated position and the actuated position.

Alternatively or additionally to any of the examples above, the biasing member may comprise an ortho-planar spring extending between the first portion and the second portion.

Alternatively or additionally to any of the examples above, the ortho-planar spring may comprise a plurality of legs extending between the first portion and the second portion, where the plurality of legs may be circumferentially spaced about the first portion.

Alternatively or additionally to any of the examples above, the biasing member may comprise a leaf-spring having two or more legs configured to engage the second portion.

Alternatively or additionally to any of the examples above, the biasing member may comprise a plurality of leaf springs circumferentially spaced about the first portion and each leaf spring of the plurality of leaf springs may be configured to engage the second portion.

Alternatively or additionally to any of the examples above, the biasing member may comprise a first magnet at the first portion and a second magnet at the second portion.

Alternatively or additionally to any of the examples above, one or both of the first magnet and the second magnet may be a ring magnet.

Alternatively or additionally to any of the examples above, the first portion may comprise a through-hole.

Alternatively or additionally to any of the examples above, the first portion may be configured to extend around an exterior of the second portion.

In an example, a valve assembly for a medical device may include a valve stem and a valve interface assembly that may comprise an interface member coupled with the valve stem, a collar member configured to receive the valve stem, and a biasing member integrally formed with the interface member and including a plurality of legs extending to the collar member, wherein the biasing member may be configured to return the valve stem to an unactuated position after the valve stem is advanced to an actuated position.

Alternatively or additionally to any of the examples above, a lumen through the valve stem may be in fluid communication with a through-hole of the interface member.

Alternatively or additionally to any of the examples above, the interface member, the collar member, and the biasing member may be integrally formed from a single unitary material.

Alternatively or additionally to any of the examples above, the biasing member and the interface member may be formed from a monolithic single material and the biasing member may be configured to engage the collar member as the interface member adjusts between the unactuated position and the actuated position.

Alternatively or additionally to any of the examples above, the interface member may be configured to extend around an exterior of the collar member.

Alternatively or additionally to any of the examples above, the valve assembly may further comprise a valve body having a gas inlet passage and a gas outlet passage, and wherein the valve stem may be configured to translate within the valve body between the actuated position and the unactuated position.

In an example, a valve assembly for a medical device may include a valve stem and a valve interface assembly that may comprise an interface member coupled with the valve stem, a collar member configured to receive the valve stem, and a biasing member comprising a first magnet integrally formed with the interface member and a second magnet at the collar member, wherein the biasing member may be configured to return the valve stem to an unactuated position after the valve stem is advanced to an actuated position.

Alternatively or additionally to any of the examples above, the second magnet may be integrally formed with the collar member.

Alternatively or additionally to any of the examples above, one or both of the first magnet and the second magnet may be ring magnets extending circumferentially around the valve stem.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a schematic perspective view of an illustrative valve;

FIG. 4A depicts a schematic cross-section view of an illustrative valve assembly, with a valve in a first configuration;

FIG. 4B depicts a schematic cross-section view of the illustrative valve assembly of FIG. 4A, with the valve in a second configuration;

FIG. 5 depicts a schematic top view of an illustrative interface assembly for a valve;

FIG. 6A depicts a schematic side view of the illustrative interface assembly of FIG. 5, with the interface assembly in a first configuration;

FIG. 6B depicts a schematic side view of the illustrative interface assembly of FIG. 5, with the interface assembly in a second configuration;

FIG. 7A depicts a schematic cross-section view of a portion of an illustrative valve assembly, with a valve in a first configuration;

FIG. 7B depicts a schematic cross-section view of the illustrative valve assembly of FIG. 7A, with the valve in a second configuration;

FIG. 8A depicts a schematic cross-section view of a portion of an illustrative valve assembly, with a valve in a first configuration;

FIG. 8B depicts a schematic cross-section view of the illustrative valve assembly of FIG. 8A, with the valve in a second configuration;

FIG. 9A depicts a schematic cross-section view of a portion of an illustrative valve assembly, with a valve in a first configuration;

FIG. 9B depicts a schematic cross-section view of the illustrative valve assembly of FIG. 9A, with the valve in a second configuration;

FIG. 10A depicts a schematic cross-section view of a portion of an illustrative valve assembly, with a valve in a first configuration; and

FIG. 10B depicts a schematic cross-section view of the illustrative valve assembly of FIG. 9A, with the valve in a second configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, the particular feature, structure, or characteristic may be effected in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. The numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element.

The detailed description is intended to illustrate but not limit the disclosure. The various elements described may be arranged in various combinations and configurations without departing from the scope of the disclosure. The detailed description illustrates example configurations of the disclosure.

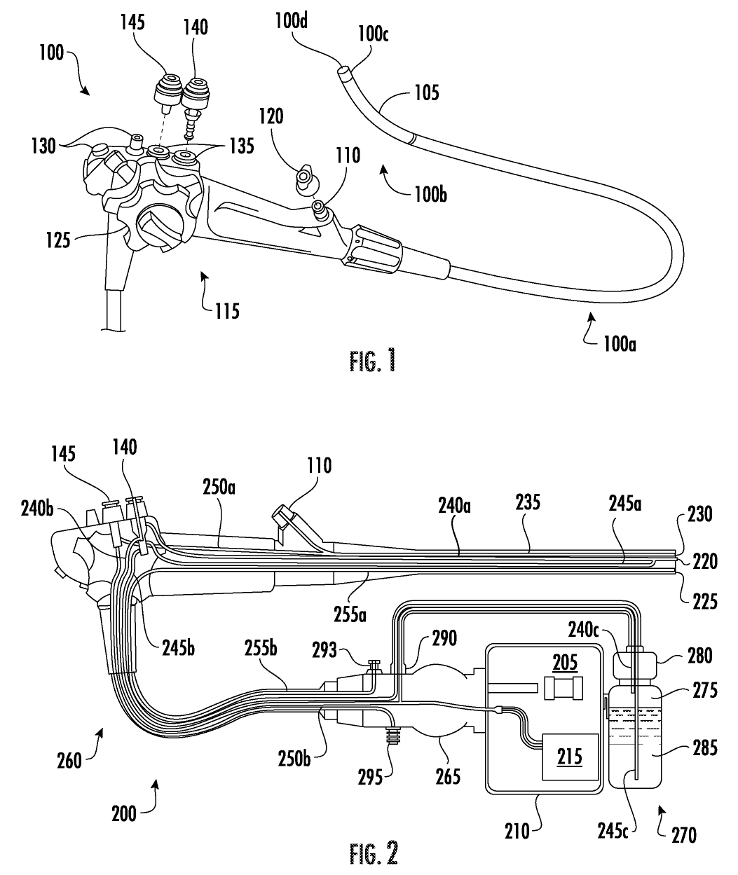

With reference to FIG. 1, an illustrative endoscope 100 is schematically depicted and FIG. 2 schematically depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serve as a component of an air/water feed circuit by housing a pressurizing pump (e.g., an air pump 215), such as an air feed pump, in the unit 210.

The endoscope shaft 100a may include a distal tip 100c (e.g., a distal tip unit adapted to be inserted into a body cavity of a patient) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the flexible bending portion 105 (e.g., one knob controls up-down steering and another knob controls left-right steering). One or more (e.g., one, two, a plurality, etc.) of video switches 130 for remotely operating the video processing unit 210 may be arranged on a proximal end side of the handle 115.

The handle 115 may be provided with dual valve locations 135. One of the valve locations 135 may have or receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220, as depicted in FIG. 2.

The other valve location 135 may have or receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO2) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water container or reservoir 270 (e.g., a water bottle and/or other suitable reservoir or container) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

Referring to FIG. 2, an illustrative operation of an endoscope system 200, including an endoscope such as the endoscope 100 depicted in FIG. 1, is explained. Air from the air pump 215 in the video processing unit 210 may flow through the connector portion 265 and branch to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user’s finger on the valve, air is allowed to flow out of the valve 140 to atmosphere. In a first position, the user’s finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve 140, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some configurations, outflow check valves or other one-way valve configurations (not shown) may be placed in the path of the lens wash supply tubing to help prevent water from back-flowing into the reservoir 270 after the water has passed the valve.

A relatively higher flow rate compared to lens wash is typically required for irrigation water, since a primary use is to clear the treatment area in the patient of debris that obstructs the user’s field of view. Irrigation is typically achieved with the use of a pump (e.g., peristaltic pump), as described. In configurations with an independent water source for irrigation, tubing placed in the bottom of a water source may be passed through the top of the water source and threaded through the head on the upstream side of the pump. Tubing on the downstream side of the pump is connected to the irrigation feed line 255b in the umbilical 260 and the irrigation supply line 255a of endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical 260, and down the irrigation supply line in the shaft 100a of the endoscope 100 to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some configurations, outflow check valves or other one-way valve configurations (not shown), similar to the lens wash tubing 245c, may be placed in the path of the irrigation supply tubing to help prevent back-flow into the reservoir after water has passed the valve.

The suction valve 145 may be configured to allow or prevent suction and/or a suction effect in the working channel 235. When the suction valve 145 is in a valve closed position (e.g., a first configuration), a suction fluid flow through the working channel 235 may be blocked by the suction valve 145. When suction is desired in the working channel 235, an operator or user may actuate the suction valve 145 (e.g., by depressing a button on the valve and/or actuating the suction valve 145 in one or more other suitable manners) in order to bring the suction valve 145 to a valve open position (e.g., a second configuration). When the suction valve 145 is in the valve opened position, a flow channel inside the suction valve 145 may connect the working channel 235 to the suction device coupled to suction connection 295 and the suction device may create a negative pressure that draws fluid into and out of the working channel 235 through an outlet provided in the suction valve. When the operator or user releases the suction valve 145, the valve 145 may return to its valve closed position and reduce or block a suction fluid flow from the working channel 235.

In some cases, suction valves 145 may rely on a path of least resistance to direct a suction fluid flow through the endoscope system 200. In some cases, when a suction pump is turned on for a procedure, the pump remains on for an entirety of the procedure and continually pulls air from the flexible umbilical 260, which in turn draws fluid from the line side of the endoscope 100 that runs up the umbilical 260 and connects to a port at the suction valve 145. When the suction valve 145 is in a first position and/or configuration (e.g., a closed position) the suction force or negative pressure from the suction pump is blocked from the working channel 235 and may pull fluid from atmosphere through the suction valve 145. When the suction valve 145 is actuated to a second position and/or configuration (e.g., an opened position) (e.g., when the button or cap associated with the suction valve 145 is depressed and/or actuated in one or more other suitable manners), the opening from atmosphere through the suction valve 145 to the suction pump may be effectively closed or blocked by the suction valve 145 and a fluid path between working channel 235 and the suction pump through the suction valve 145 may be opened. Thus, fluid moving to the suction pump may follow a path of least resistance, where the path may change depending on whether the suction valve 145 is in a first position (e.g., a closed position) or a second position (e.g., an opened position)

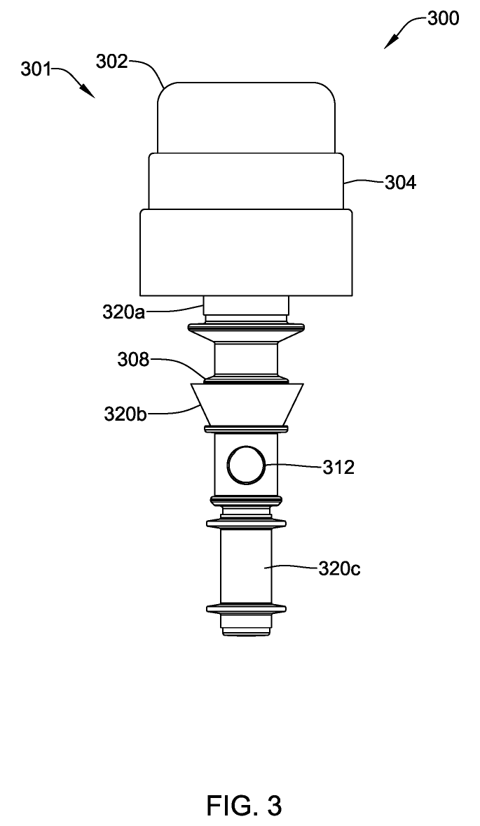

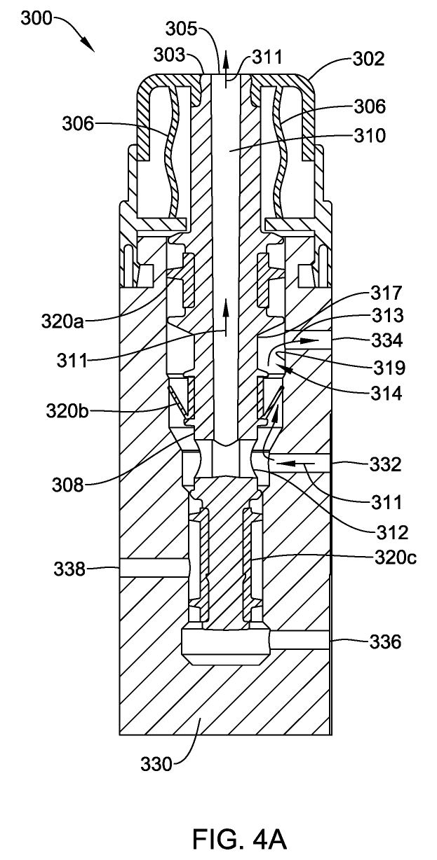

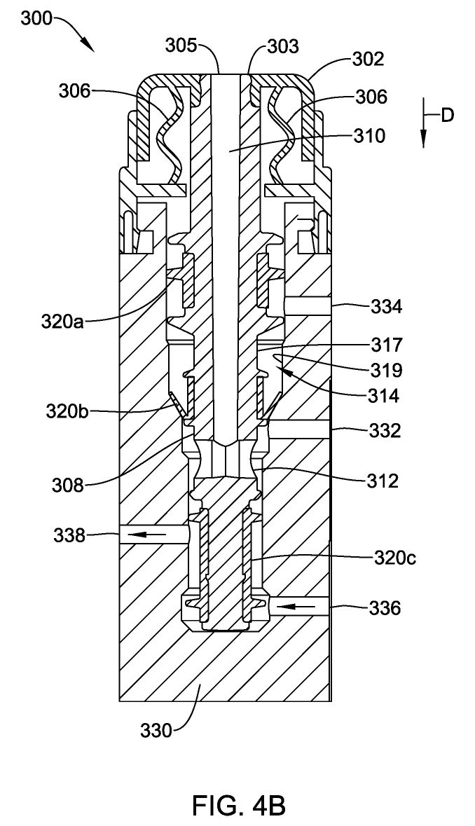

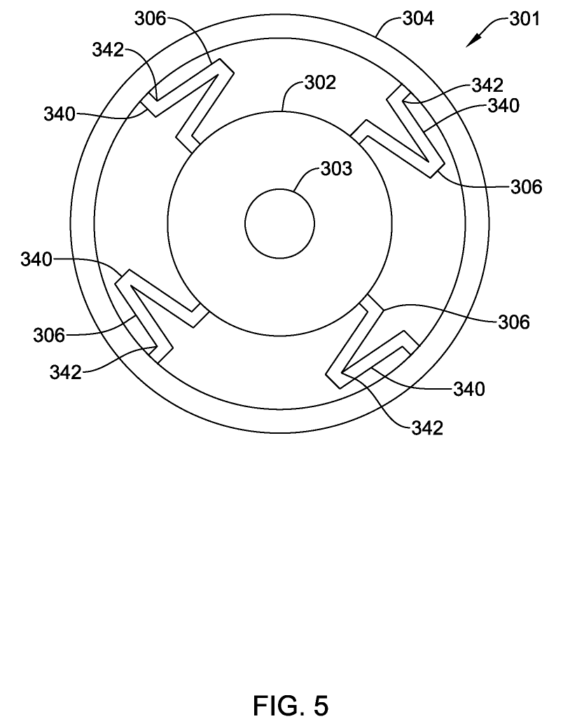

FIG. 3 schematically depicts an illustrative configuration of a valve assembly or valve 300 (hereinafter “valve”) that may be used as a gas/water valve 140, a suction valve 145, and/or other suitable type of valve in the endoscope system 200 and/or other suitable system. FIGS. 4A and 4B schematically depict cross-section views of the valve 300 depicted in FIG. 3 positioned within a valve body 330. In some examples, the valve body 330 may be part of the valve assembly.

Among other suitable components and/or features, the valve 300 may include a valve interface assembly 301 and a valve stem 308. In some examples, the interface assembly 301 may include an interface component or member 302 (e.g., a first portion), a collar component or member 304 (e.g., a second portion), a biasing component or member 306 (e.g., a third portion), as depicted for example in FIGS. 4A and 4B, and/or other suitable components or features. The interface member 302 may include one or more openings 303 (e.g., a gas escape hole and/or other suitable opening) and the valve stem 308 may include a central lumen 310 extending between one or more first openings 305 (e.g., where one or more first openings 305 may be in communication (e.g., fluid communication) with at least one of the one or more openings 303 of the interface member 302) and one or more second openings 312 (e.g., a gas inlet passage), as depicted for example in FIGS. 4A and 4B.

The biasing member 306 may be configured in any suitable manner configured to bias the interface member 302 and/or the valve stem 308 in a proximal direction or an unactuated position relative to the collar member 304. In some examples, the biasing member 306 may be or more include one or more springs, bellows, ortho-planar springs, leaf springs, magnetic biasing members, electrical biasing members, and/or other suitable components configured to bias the interface member 302 to an unactuated position. In one example, the biasing member 306 may be or may include one or more ortho-planar spring with a plurality of legs extending between the interface member 302 and the collar member 304. In one example, the biasing member may be or may include one or more leaf springs. In one example, the biasing member 306 may be or may include one or more magnetic biasing components. Other suitable configurations of the biasing member 306 are contemplated.

The valve stem 308 may couple to the interface member 302 in any suitable manner. In some cases, a portion (e.g., a proximal portion) of the valve stem 308 at or proximate to the opening 305 may be coupled to the interface member 302 via one or more suitable coupling mechanisms. Example suitable coupling mechanisms include, but are not limited to, adhesive, a threaded connection, a luer lock connection, a snap connection, a ball-detent connector, a friction fit, and/or additional or alternative coupling mechanisms.

The components of the interface assembly 301 may be formed in any suitable manner. For example, all or at least two or more of the components or portions of the interface assembly 301 may be formed monolithically of a single material, one or more components or portions of the interface assembly 301 may be formed separate from forming one or more other components or portions of the interface assembly 301, one or more components or portions of the interface assembly 301 may be embedded in one or more other components or portions of the interface assembly 301, and/or one or more components or portions of the interface assembly 301 may be formed in one or more other suitable manners.

Any suitable techniques may be utilized to form the components or portions of the interface assembly 301. In some examples, though not required, the components or portions of the interface assembly 301 may be formed using one or more of a molding process, an injection molding process, an over molding process, a casting process, a finishing process, sanding, and/or by or with one or more additional or alternative manufacturing techniques. In one example, the components or portions of the interface assembly 301 may be formed using an injection molding process.

The valve stem 308 may have any suitable configuration. In some examples, the valve stem 308 may be configured to adjust positions within the valve well, adjust flow paths to the gas and liquid supplies and feeds, and couple to the interface member 302.

The valve stem 308 may be formed in any suitable manner. In some examples, though not required, the valve stem 308 may be formed using a molding process, an injection molding process, an over molding process, a casting process, a finishing process, sanding, and/or by or with one or more additional or alternative manufacturing techniques. In one example, the valve stem 308 may be formed using an injection molding process.

The valve 300 may include any suitable number of seals along the valve stem 308. In some examples, three seals 320a, 320b, 320c may extend circumferentially around (e.g., extend at least partially or entirely circumferentially around) the valve stem 308 (e.g., around the elongate body of the valve stem 308) at different axial locations. In one example, a first seal 320a may be located between the first opening 305 and the second opening 312 at a location configured to be proximal of the gas outlet passage 334 of the valve body 330 to obstruct flow in the valve 300 proximal the location of the gas outlet passage 334, a second seal 320b may be located between the first seal 320a and the second opening 312 and may be configured to not impede gas flow when the valve stem 308 is in the second configuration, and a third seal 320c may be located between a distal end of the valve stem 308 and the gas inlet 332, but other suitable configurations are contemplated. Alternatively or additionally, the seals of the valve 300 may be located along the valve body 330 such that the valve stem 308 may move relative to the seals.

The seals 320a, 320b, 320c may have any suitable configuration. In some examples, each of the seals 320a, 320b, 320c may include on one or more wipers or flanges configured to engage the inner surface 319 of the valve body 330. In one example, the first seal 320a may have a single wiper or flange, the second seal 320b may have a single wiper or flange, and the third seal 320c may have two wipers or flanges, but other suitable configurations are contemplated.

The components of the valve 300 may be formed from any suitable materials. For example, the interface assembly 301, the valve stem 308, and the seals 320a, 320b, 320c may be formed from the same materials or different materials.

The components or portions of the interface assembly 301 may be formed from any suitable material. For example, the components of the interface assembly 301 may be formed from material including, but not limited to, metals, polymer, plastic, bioplastic, recyclable materials, acrylonitrile butadiene styrene (ABS), polycarbonate, thermoplastic elastomers (TPE), thermoplastic polyurethane (TPU), liquid silicone rubber (LSR), steel, aluminum, and/or other suitable material. In one example, interface member 302, the collar member 304, and the biasing member 306 may be formed from a single material, such as a bioplastic or other suitable material. In another example, the interface member 302 and the biasing member 306 may be formed from a single material, such as a bioplastic or other suitable material, and the collar member 304 may be formed from one or more other suitable materials.

The valve stem 308 and the seals 320a-320c may be formed from any suitable materials. In some examples, the valve stem 308 may be formed from a first material and seals 320a-320c of or coupled with the valve stem 308 may be formed from a second material, where the second material may be the same as or different than the first material. The valve stem 308 may be formed from a hard or rigid polymer and all of, part of one or more of, or one or more of the seals 320a-320c may be formed from a flexible polymer, but other suitable configurations are contemplated. In some examples, the valve stem 308 may be formed from polymer, acrylonitrile butadiene styrene (ABS), polycarbonate, and/or other suitable material. Alternatively, the valve stem 308 may be formed of steel or aluminum. The seals 320a-320c may be formed from one or more of a polymer, thermoplastic elastomers (TPE), thermoplastic polyurethane (TPU), liquid silicone rubber (LSR), and/or other suitable materials.

The material of the seals 320a, 320b, 320c may have any suitable durometer. In one example, the material of the seals 320a, 320b, 320c when positioned at the valve stem 308 may have a durometer in a range of about 20-80 shore A, about 30-60 shore A, and/or other suitable values within one or more other suitable ranges of durometer, but could be softer or firmer depending on the geometry used for the seals and the amount of interference desired with the inner surface 319 of the valve body 330 (e.g., depicted in FIGS. 4A and 4B). In one example, the seals 320a, 320b, 320c may be formed from silicone with a durometer in a range of 40-50 shore A, but this is not required.

As depicted for example in FIGS. 4A and 4B, the valve 300 may be inserted into the valve body 330 (e.g., one of the valve locations 135 described with respect to FIGS. 1 and 2). The valve body 330 may be sized and shaped to receive the valve stem 308, as well as alternative valve designs. In some examples, the valve body 330 may be for gas/lens operations, as depicted, or for suction operations. The valve body 330 may include a gas inlet passage 332 (e.g., an air inlet passage) communicating with a source of gas (e.g., air, etc. as described above with respect to the gas supply line 240a). A gas outlet passage 334 (e.g., an air outlet passage) in the valve body 330 may communicate with a gas feed line (e.g., the gas feed line 240b and/or other suitable gas line). The valve body 330 may include a liquid inlet passage 336 (e.g., a water inlet, etc.) connected to or otherwise in communication with a liquid supply (e.g., a water supply) and a liquid outlet passage 338 (e.g., a water outlet, etc.) connected to or otherwise in communication with a liquid feed line.

Gas flowing into the valve body 330 via the gas inlet passage 332, may travel along the path of least resistance and as such, the gas may flow along a first flow path 311 and out the first opening 305 when the first opening 305 is unblocked (e.g., the valve 300 is in a first configuration) and along a second flow path 313 (e.g., the valve 300 is in a second configuration) across the second seal 320b and out of the gas outlet 334 when the first opening 305 is blocked. FIG. 4 depicts the interface member 302 in the unactuated position and, although the first opening 305 is unblocked in FIG. 4A, the first flow path 311 and the second flow path 313. Further, although FIG. 4A schematically depicts the second seal 320b blocking the second flow path 313, pressure within the valve 300 may cause the second seals 320b to deflect open to allow flow through the gas outlet passage 334 when the first opening 305 is covered.

FIG. 4B schematically depicts a third configuration of the valve 300 in which the valve stem 308 has been advanced distally relative to the valve body 330 in response to actuation of the interface member 302 in a distal direction D (e.g., in response to a force acting on the interface member 302 in the distal direction D) to an actuated position. Although not depicted in FIG. 4B, a user may cover the first opening 305 when the valve 300 is in the third configuration. In some examples, the biasing member 306 extending between the interface member 302 and the collar member 304 may be compressed as the interface member 302 is moved in the distal direction to the actuated position. When the force in the distal direction D is removed from the interface member 302, the biasing member 306 may return the interface member 302 and the valve stem 308 to the first or second configuration. In the third configuration, as depicted for example in FIG. 4B, the space 314 is no longer in fluid communication with the gas inlet passage 332 in the valve body 330. Rather, the second seal 320b (e.g., a middle seal or other suitably positioned seal) may be seated along the inner wall of the body 330 to obstruct or prevent gas flow proximal of the gas inlet passage 332.

When the valve stem 308 is in the third configuration, the two wipers or flanges of the third seal 320c may be positioned such that a distal wiper or flange of the third seal 320c is distal of the liquid inlet passage 336 and a proximal wiper or flange of the third seal 320c is proximal of the liquid outlet passage 338, which may create an annular passage between the valve stem 308 and valve body 330 in which liquid may flow from the liquid inlet passage 336 to the liquid outlet passage 338 along a third flow path 315. Upon release of the force in the distal direction D and return of the interface member 302 and valve stem 308 to respective positions associated with the first or second configurations, the placement of the third seal 320c may block or prevent additional liquid from entering the space between the axially spaced wipers or flanges of the third seal 320c. In the first, second, or third configurations of the valve 300, the placement of the third seal 320c on the valve stem 308 may prevent liquid from entering gas flow paths.

Endoscope valves may be formed from a plurality of parts including a cap, one or more springs (e.g., metal springs), and a collar. Because of how the components are coupled, gloves, gowns, and/or other equipment may be caught in the valves (e.g., in the spring, etc.), which may inadvertently cause contamination of a procedural environment. Further, gas/water valves and suction valves for endoscopes may be intended for single use to prevent or mitigate infections or contamination through using the valves in multiple procedures. Single use valve components, however, create material and/or financial waste during manufacture and/or after use, which is amplified when the single use components are formed from multiple components.

The concepts discussed herein include illustrative configurations of the interface assembly 301 for the valve 300 (e.g., the air/water valve, the suction valve, etc.) that may mitigate waste by mitigating the number of components used to form the valve 300 (e.g., a single-use valve). In one example, an illustrative configuration of the interface assembly 301 may include the interface member 302 integrally formed with the biasing member 306 from a unitary or monolithic single material, as depicted for example in FIGS. 4A and 4B. In one example, an illustrative configuration of the interface assembly 301 may include the interface member 302, the collar member 304, and the biasing member 306 integrally formed from a monolithic single material. As such, valves 300 may include interface assemblies 301 having as little as a single component or two components. In some examples, the interface assemblies 301 may be partially or entirely formed from one or more bioplastics or recyclable plastics, which may contribute to producing a sustainable and/or environmentally friendly single use valve.

FIG. 5 depicts a schematic top view of an illustrative configuration of the interface assembly 301 with all components of the interface assembly 301 formed from a monolithic single material. In one example, the biasing member 306 may be an ortho-planar spring having a plurality of legs 340 extending between the interface member 302 and the collar member 304, but other suitable configurations of the biasing member 306 are contemplated when the interface member 302, the collar member 304, and the biasing member 306 are formed from a monolithic single material.

The legs 340 of the biasing member 306 may have any suitable configuration. In some examples, the legs 340 may have one or more flex-locations 342 (not all flex-locations 342 are labeled for clarity reasons) and may extend radially outward from and perpendicular to or at least transverse to an axis of a direction of movement of the interface member 302 when the interface member 302 is adjusted between an unactuated position and an actuated position. Although the legs 340 may have a V-shape with a plurality of flex-locations 342, as depicted for example in FIG. 5, the legs 340 may take on any other suitable configuration that promotes resiliency of the legs 340.

The flex-locations 342 may have any suitable configuration and the legs 340 may have any suitable number of flex-locations 342. Although FIG. 5 depicts the legs 340 with three flex-locations, each leg 340 may have fewer than three flex-locations 342 or more than three flex-locations 342. In some examples, the flex-location 342 may be locations in the leg configured to bend, locations in the legs 340 that are thinner than other locations, and/or the flex-locations 342 of the legs 340 may have one or more other suitable configurations.

As depicted for example in FIG. 5, the legs 340 may be biased to a bent configuration and when the interface member 302 is actuated from an unactuated position to an actuated position, the legs 342 may straighten or unbend at the flex-locations 342. Alternatively or additionally, one or more of the legs 340 may be biased to be straight in an unactuated position and adjust to an actuated position in which the one or more of the legs 340 are bent.

The legs 340 may have any suitable length. In some examples, a length of the legs 340 may be determined to allow the interface member 302 to translate longitudinally a predetermined or desired distance between the unactuated position and the actuated position.

Any suitable number of legs 340 may be utilized. In one example and as depicted in FIG. 5, four legs 340 may be utilized. However, the biasing member 306 configured as an ortho-planar spring may have fewer than four legs 340 or more than four legs 340. In some examples, the number of legs 340 and/or a configuration of each leg 340 may be adjusted to modify a spring force and/or total displacement length of the interface member 302 between the unactuated position and the actuated position. In one example, when fewer legs 340 are utilized for the biasing member 306, longer levers (e.g., segments between flex-locations 342) may be utilized to increase the total displacement length relative to when more legs 340 are utilized. In on example, utilizing more legs 340 may increase an amount of force needed to adjust the interface member longitudinally between the unactuated position and the actuated position while limiting the total displacement length due to having shorter levers.

As depicted in FIG. 5, the legs 340 may extend between an outer surface of the interface member 302 and the collar member 304 and the interface member 302 may extend within an interior of the collar member 304. Alternatively or additionally, the interface member 302 may extend around an exterior of the collar member 304 at least in the actuated configuration. When the interface member 302 is configured to extend around an exterior of the collar member 304, one or more legs 340 may extend between an interior of the interface member 302 and an exterior of the collar member 304. Other suitable configurations of the interface assembly 301 are contemplated.

When the biasing member 306 configured as an ortho-planar spring includes a plurality of legs 340, the legs 340 may be circumferentially spaced about the interface member 302 and/or between the interface member 302 and the collar member 304. In some examples, the legs 340 may be equally circumferentially spaced from one another, as depicted for example in FIG. 5, but other suitable spacing of the legs 340 is contemplated.

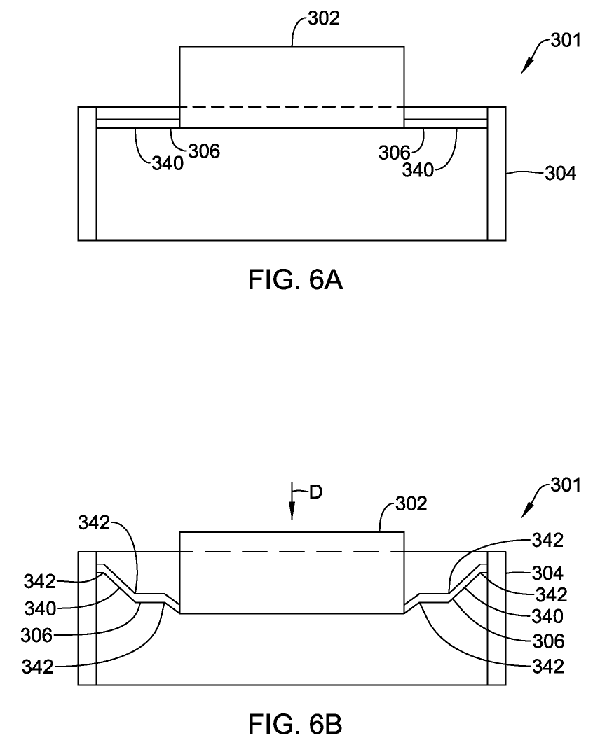

FIGS. 6A and 6B depict schematic side views of the configuration of the interface assembly 301 depicted in FIG. 5, with the collar member 304 being transparent so as to render a base of the interface member 302 and the legs 340 of the biasing member 306 viewable through the collar member 304. As depicted in FIGS. 6A and 6B, in some examples, the biasing member 306 (e.g., the legs 340 of the biasing member 306) may extend between a bottom of the interface member 302 and a top of the collar member 304, but other suitable configurations are contemplated.

FIG. 6A schematically depicts the interface member 302 in an unactuated position or configuration. When the interface member 302 is in the unactuated state, the biasing member 306 (e.g., the legs 340) may be in a relaxed state. In one example, when in the relaxed state, the legs 340 may be bent and extend perpendicular to or at any other suitable transverse angle with an axis of travel of the interface member 302 as the interface member 302 is actuated. Other suitable configurations of the biasing member 30 are contemplated.

FIG. 6B schematically depicts the interface member 302 in an actuated position or configuration. When the interface member 302 is in the actuated state, the biasing member 306 (e.g., the legs 340) may be in a stressed state. In one example, when in the stressed state, the legs 340 may be straightened, the legs 340 may extend longitudinally, and the legs 340 may be at a tighter angle with respect to the axis of travel of the interface member 302 as the interface member 302 is actuated. When a force in the distal direction D applied to the interface member 302 to actuate the interface member 302 is removed from the interface member 302, the legs 340 of the biasing member 306 may return to the relaxed state to return the interface member 302 to the unactuated position. Although the legs 340 may include one or more flex-locations 342, a risk of gloves, gowns, and/or other procedural equipment being unintentionally caught in the biasing member 306 may be mitigated with the configuration of the biasing member 306.

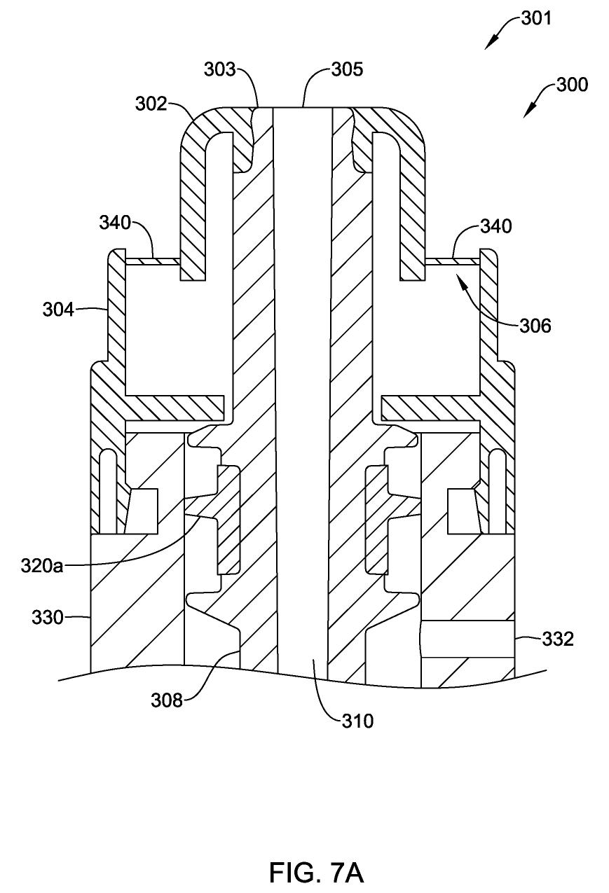

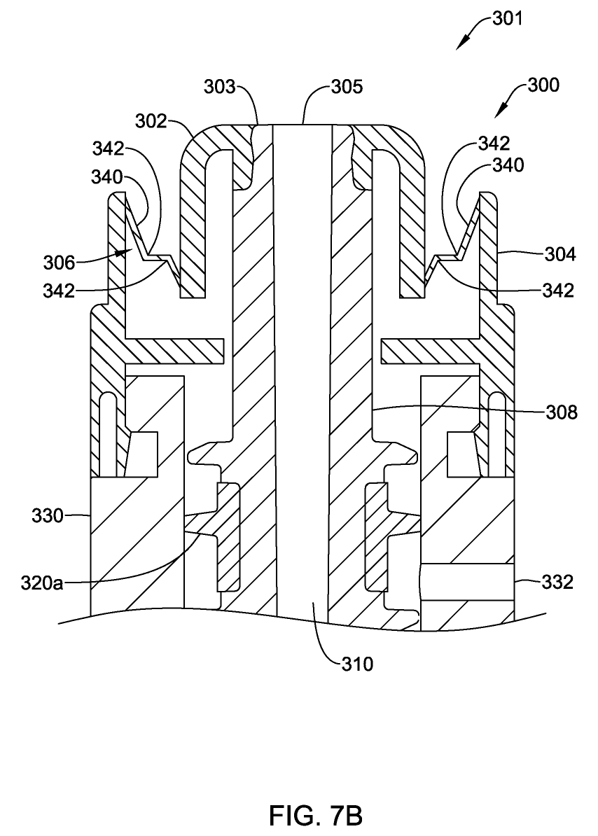

FIGS. 7A and 7B depict schematic partial cross-section views of the valve 300 within the valve body 330, where the valve 300 includes an interface assembly 301 having the biasing member 306 configured as an ortho-planar spring. FIG. 7A depicts the interface member 302 is in the unactuated position with the valve stem 308 in the first configuration or second configuration. When the interface member 302 is in the unactuated position, the legs 340 may be in the relaxed state and may be substantially perpendicular to a direction of travel of the interface member 302 and/or the valve stem 308. FIG. 7B depicts the interface member 302 in the actuated position with the valve stem 308 in the third configuration. When the interface member 302 is in the actuated position, the legs 340 may be in a stressed state and may be extended longitudinally in a direction of travel of the interface member 302 and/or the valve stem 308.

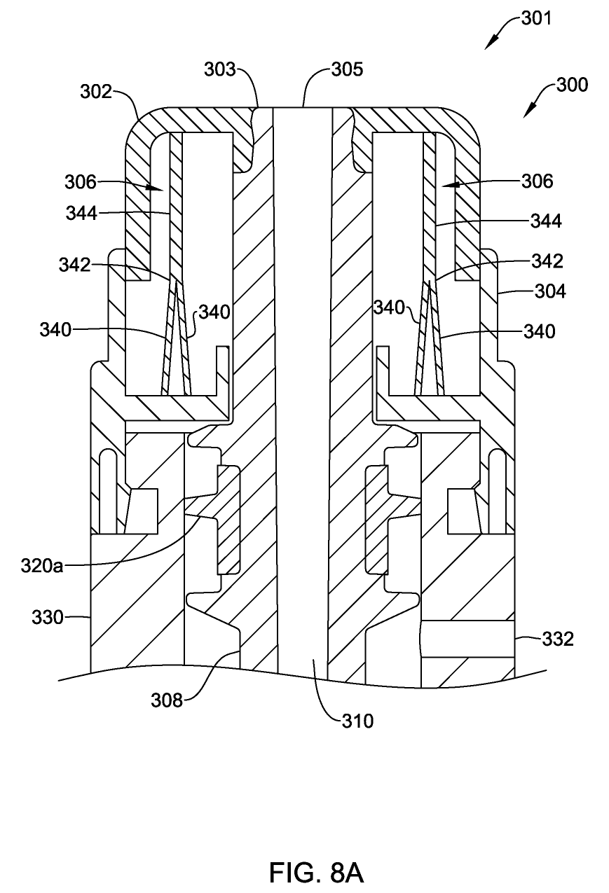

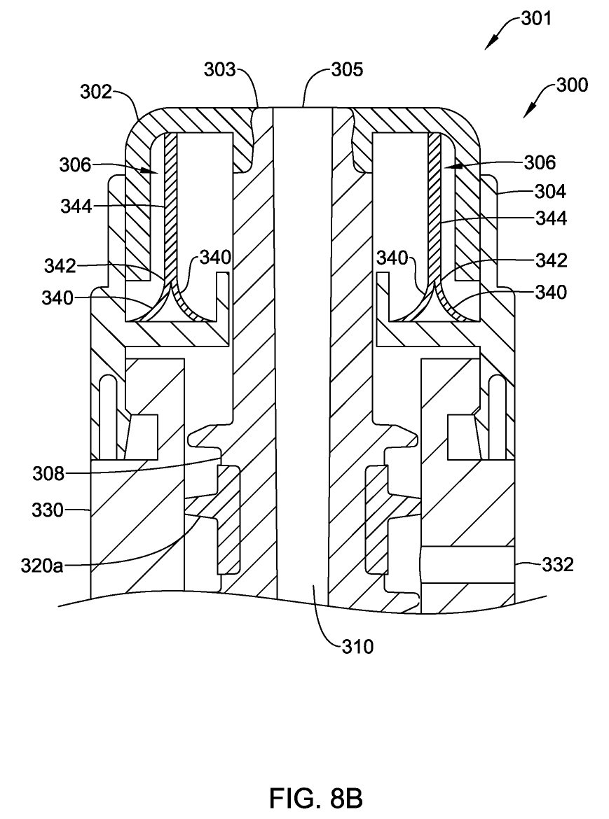

FIGS. 8A and 8B depict schematic partial cross-section views of the valve 300 within the valve body 330, where the valve 300 may include the interface assembly 301 having the biasing member 306 configured as one or more leaf springs. As depicted in FIGS. 8A and 8B, the interface member 302 and the biasing member 306 may be formed from a monolithic single material. In one example, each leaf spring of the biasing member 306 may have a body 344 extending from the interface member 302 and one or more legs 340 (e.g., one leg 340, two or more legs 340, etc.) extending from the body 344 and configured to engage a fixed surface relative to the biasing member 306. In some examples, the fixed surface configured to engage with the legs 340 of the leaf spring may be a surface of the collar member 304, but other suitable fixed surfaces of the valve assembly are contemplated to be suitable to engage the legs 340. Although the biasing member 306 is depicted in FIGS. 8A and 8B as being formed with the interface member 302 and extending away from the interface member 302, the biasing member 306 and the collar member 304 may be formed as a monolithic single material with the body 344 extending from the fixed surface of the collar member 304 and the legs 340 extending from the body 344 and configured to engage an inner surface of the interface member 302 as the interface member 302 adjusts between the unactuated position and the actuated position.

The legs 340 of the biasing member 306 configured as a leaf spring may have any suitable configuration. In some examples, the legs 340 may have one or more flex-locations 342 (not all flex-locations 342 are labeled for clarity reasons) at or proximate where the legs 340 join the body 344. In some examples, the legs 340 of the leaf spring may be curved to facilitate bending of the legs 340 as the interface member 302 is depressed to adjust from the unactuated position to the actuated position. In some examples, when the relaxed state, the legs 340 may be extend radially outward from the body 344 to facilitate further extension outward as the interface member 302 is depressed. Although the plurality of legs 340 may have a V-shape with an adjacent leg 340, as depicted for example in FIG. 8A, the legs 340 may take on any other suitable configuration that promotes resiliency of the legs 340. As depicted for example in FIG. 8A and 8B, the legs 340 of the biasing member 306 may be biased to a straight or a less bent configuration and when the interface member 302 is actuated from an unactuated position to an actuated position, the legs 340 may be bent further at the flex-locations 342 and/or along a length of the legs 340.

The legs 340 and/or the body 344 may have any suitable length. In some examples, a length of the legs 340 may be determined to allow the interface member 302 to translate longitudinally a predetermined or desired distance between an unactuated position and an actuated position.

Any suitable number of legs 340 may be utilized. In one example and as depicted in FIGS. 8A and 8B, each leaf spring of the biasing member 306 may include two legs 340. However, the leaf springs of the biasing member 306 have a single leg 340 or more than two legs 340. In some examples, the number of legs 340 and/or a configuration of each leg 340 may be adjusted to modify a spring force of the biasing member 306. In one example, when fewer legs 340 are utilized for each leaf spring, the biasing member 306 may have a lower spring force and when more legs 340 are utilized for each leaf spring, the biasing member 306 may have a greater spring force.

Any suitable number of leaf springs may be utilized as the biasing member 306 extending between the interface member 302 and the collar member 304. In one example and as depicted in FIGS. 8A and 8B, the biasing member 306 may include two leaf springs. However, the biasing member 306 may have a single leaf spring or more than two leaf springs. In some examples, the number of leaf springs may be adjusted to modify a spring force of the biasing member 306. In one example, when fewer leaf springs are utilized, the biasing member 306 may have a lower spring force and when more leaf springs are utilized, the biasing member 306 may have a greater spring force.

When a plurality of leaf springs is utilized as the biasing member 306, the leaf springs may be circumferentially spaced about the interface member 302. Although other suitable configurations are contemplated, the leaf springs may be equally circumferentially spaced from one another around the interface member 302.

As depicted in FIGS. 8A and 8B, the interface member 302 may extend within an interior of the collar member 304 when the biasing member 306 is configured as one or more leaf springs. Alternatively or additionally, the interface member 302 may extend around an exterior of the collar member 304 at least in the actuated configuration. Other suitable configurations of the interface assembly 301 are contemplated.

As depicted in FIG. 8A, when the interface member 302 is in the unactuated position, the valve stem 308 may be in the first configuration or the second configuration. When the interface member 302 is the in unactuated position, the legs 340 may be in the relaxed state and may be or have a portion that is substantially parallel to a direction of travel of the interface member 302 and/or the valve stem 308. FIG. 8B depicts the interface member 302 in the actuated position with the valve stem 308 in the third configuration. When the interface member 302 is in the actuated position, the legs 340 may be in a stressed state and may be extended, bent, or curved radially outward or in one or more manners that is in a direction transverse to a direction of travel of the interface member 302 and/or the valve stem 308.

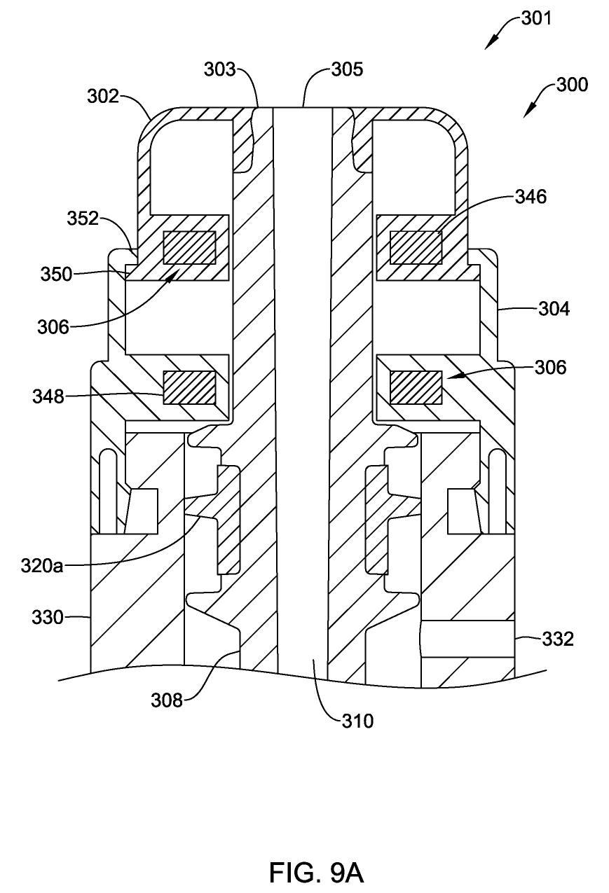

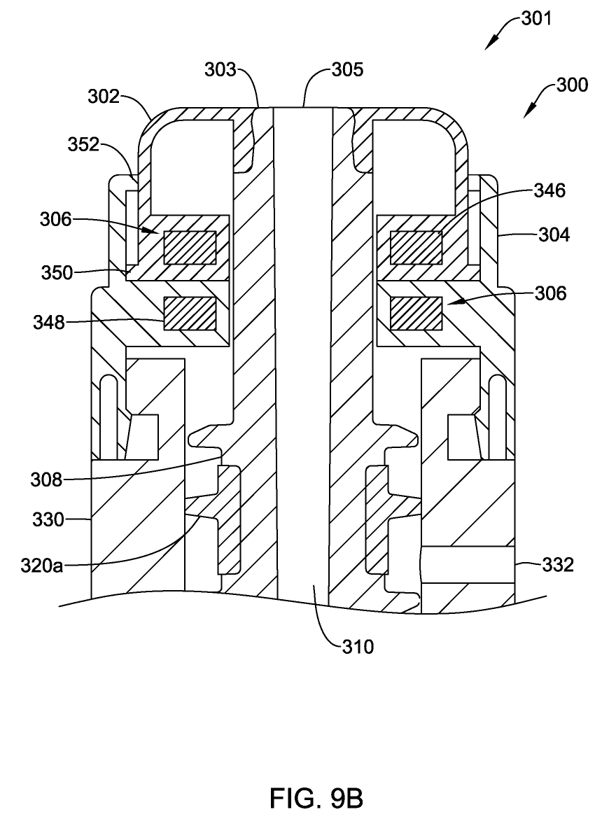

FIGS. 9A and 9B depict schematic partial cross-section views of the valve 300 within the valve body 330, where the valve 300 includes an interface assembly 301 having an illustrative configuration of the biasing member 306 that utilizes magnetic opposition to bias the interface member 302 to an unactuated position. As depicted in FIGS. 9A and 9B, one or more first magnets 346 of the biasing member 306 may be located at or coupled with the interface member 302 and aligned with one or more second magnets 348 that may be located at or coupled with the collar member 304. Although the one or more second magnets 348 are depicted as being at the collar member 304, the one or more second magnets 348 may be located at or coupled with one or more other components of the valve 300.

The interface assembly 301 may include one or more coupling features, such as protrusions and/or indentations configured to engage one another to prevent unintentional complete-separation or unintentional over-separation of the interface member 302 from the collar member 304. In one example, the interface member 302 may include a first protrusion 350 extending radially outward and the collar member 304 may include a second protrusion 352 extending radially inward, where a proximal surface of the first protrusion 350 and a distal surface of the second protrusion 352 may interact to limit separation or movement of the interface member 302 and the collar member 304 relative to one another, as depicted for example in FIGS. 9A and 9B. Other suitable configurations of protrusions and/or indentations to prevent unintentional separation of the interface member 302 and the collar member 304 are contemplated.

The protrusions and/or indentations of the interface member 302 and the collar member 304 may be located at structures or surfaces of the interface member 302 and the collar member 304 that face one another to facilitate interaction between the respective protrusions and/or indentations. In some examples and as depicted in FIGS. 9A and 9B, the first protrusion 350 and the second protrusion 352 may extend circumferentially around the interface member 302 and the collar member 304. Alternatively or additionally, one or both of first protrusion 350 and the second protrusion 352 may have extend only a portion of the circumference of the interface member 302 and the collar member 304 or the first protrusion 350 and/or the second protrusion 352 may have sub-features spaced about the circumference of the interface member 302 and the collar member 304. In one example, the first protrusion 350 of the interface member 302 may have a plurality of circumferentially spaced apart sub-protrusions that are configured to engage the second protrusion 352 extending entirely circumferentially around the collar member 304. Other suitable configurations of the protrusions and/or indentations of the interface member 302 and the collar member 304 are contemplated.

As depicted in FIGS. 9A and 9B, the one or more first magnets 346 may be integrally formed with (e.g., embedded in) the interface member 302, but other suitable configurations are contemplated. In some examples, the interface member 302 may be formed using a molding technique and the interface member 302 may be over molded entirely or at least partially around the first magnets 346. Alternatively or additionally, the first magnets 346 may be coupled with the interface member 302 in any other suitable manner including, but not limited to, via adhesive, via a press-fit, and/or via one or more other suitable coupling techniques.

As depicted in FIGS. 9A and 9B, the one or more second magnets 348 may be embedded in the collar member 304, but other suitable configurations are contemplated. In some examples, the collar member 304 may be formed using a molding technique and the collar member 304 may be over molded entirely or at least partially around the second magnets 348. Alternatively or additionally, the second magnets 348 may be coupled with the collar member 304 and/or other portion of the valve 300 in any other suitable manner including, but not limited to, via adhesive, via a press-fit, and/or via one or more other suitable coupling techniques.

The one or more first magnets 346 and the one or more second magnets 348 may be any suitable type of magnets. In some examples, the first magnets 346 and the second magnets 348 may have the same or similar polarity so as to repel one another and bias the interface member 302 away from the collar member 304. The first magnets 346 and the second magnets 348 may be formed from any suitable type of material including, but not limited to, biocompatible magnetic materials, recyclable magnetic materials, magnetic granules embedded in polymer material, titanium, titanium alloys, cobalt-chromium alloys, and/or other suitable materials.

Any suitable number of first magnets 346 and second magnets 348 having any suitable layout may be utilized. For example, one or each of the first magnets 346 and the second magnets 348 may be formed from a single magnetic component or from a plurality of magnetic components. In some examples, the first magnets 346 may be located at a structure or surface of the interface member 302 configured to be proximate a structure or surface of the collar member 304 or other portion of the valve 300 at which the second magnets 348 may be located. In one example, the first magnet 346 may be a single ring or annular magnet extending around or about an opening or lumen in the interface member 302 for receiving the valve stem 308 (e.g., the first magnet 346 may extend around the valve stem 308) and the second magnet 348 may be a single ring or annular magnet extending around or about an opening or lumen in the collar member 304 for receiving the valve stem 308 (e.g., the second magnet 348 may extend around the valve stem 308), where the first magnet 346 and the second magnet 348 may be positioned to bias the interface member 302 to the unactuated position as depicted in FIG. 9A and increase a repelling or opposition force between the first magnet 346 and the second magnet 348 as the interface member 302 approaches the collar member 304 in the actuated position depicted in FIG. 9B. Other suitable configurations of the first magnets 346 in the interface member 302 and the second magnets 348 in the collar member 304 are contemplated.

As depicted in FIG. 9A, when the interface member 302 is in the unactuated position, the valve stem 308 may be in the first configuration or second configuration. When the interface member 302 is the in unactuated position, a magnetic field between the first magnet 346 and the second magnet 348 may be relatively weak, but still bias the interface member 302 to the unactuated position. The interaction between the first protrusion 350 and the second protrusion 352 may limit movement of the interface member 302 in a proximal direction relative to the collar member 304 in response to biasing of the magnetic fields of the first magnet 346 and the second magnet 348. FIG. 9B depicts the interface member 302 in the actuated position with the valve stem 308 in the third configuration. When the interface member 302 is in the actuated position, the magnetic field between the first magnet 346 and the second magnet 348 may be strong relative to when the interface member 302 is in the unactuated position so as to return the interface member 302 to the unactuated position when a force in the distal direction that is actuating the interface member 302 to the actuated position is removed from the interface member 302.

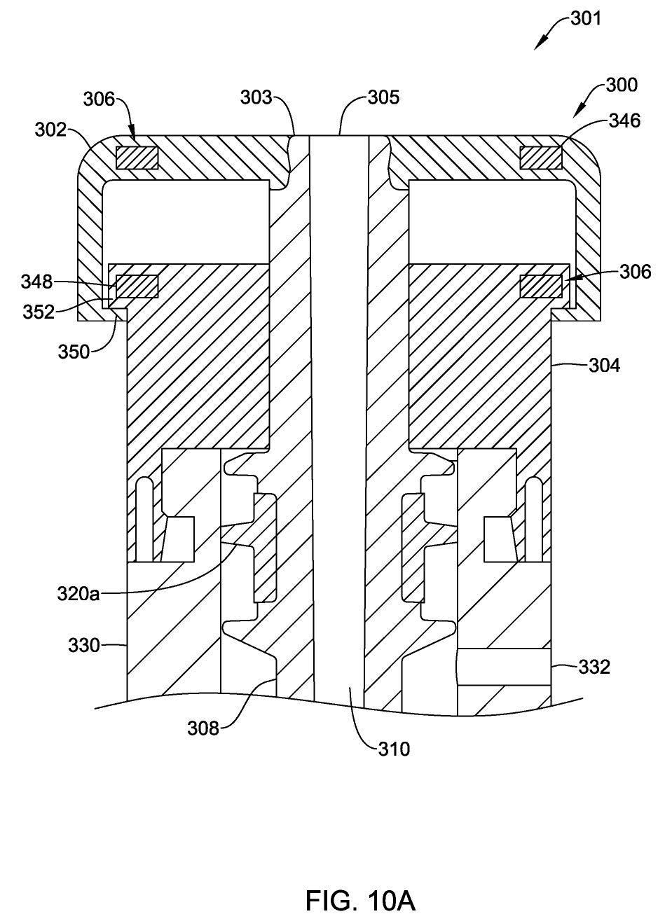

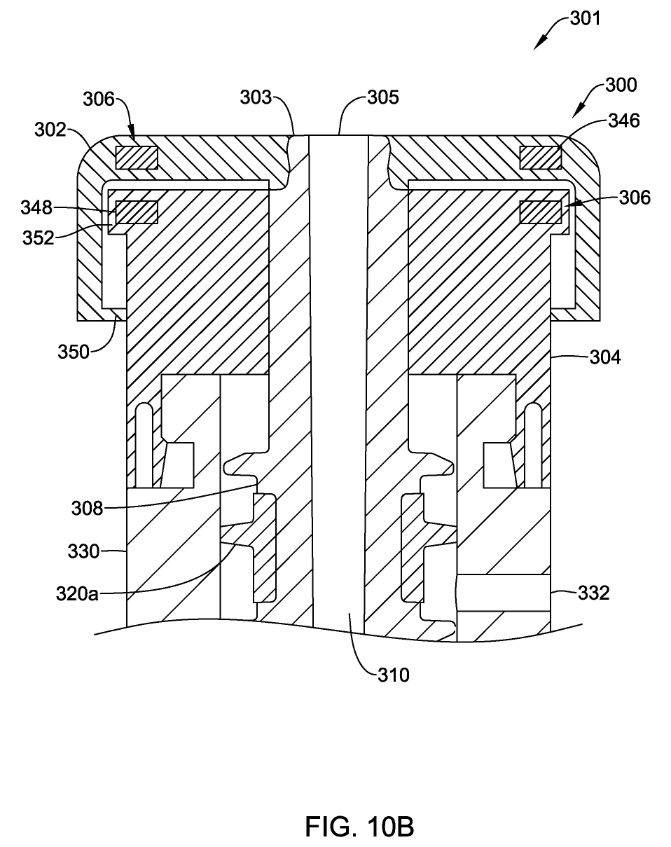

FIGS. 10A and 10B depict schematic partial cross-section views of the valve 300 within the valve body 330, where the valve 300 includes an interface assembly 301 having an illustrative configuration of the biasing member 306 that utilizes magnetic opposition to bias the interface member 302 to an unactuated position. As depicted in FIGS. 10A and 10B, one or more of the first magnets 346 of the biasing member 306 may be located at or coupled with the interface member 302 and aligned with one or more of the second magnets 348 located at or coupled with the collar member 304. Although the one or more second magnets 348 are depicted as being at the collar member 304, the one or more second magnets 348 may be located at or coupled with one or more other components of the valve 300. Similar to as discussed with respect to FIGS. 9A and 9B, the first magnet 346 may be embedded in the interface member 302 and the second magnet 348 may be embedded in the collar member 304, but other suitable arrangements are contemplated.

As depicted in the example of FIGS. 10A and 10B, the first magnet 346 may be a ring or annular magnet located in or at a proximal portion of the interface member 302 and the second magnet 348 may be a ring or annular magnet located in or at a proximal portion of the collar member 304. The positioning of the first magnet 346 and the second magnet 348 may facilitate a configuration of the interface assembly 301 in which the interface member 302 may extend around and/or along an exterior of the collar member 304, as depicted for example in FIGS. 10A and 10B. When the interface member 302 may extend around or along an exterior of the collar member 304, the first protrusion 350 of the interface member 302 may be located distal of the second protrusion 352 of the collar member 304 such that a proximal surface of the first protrusion may interact with a distal surface of the second protrusion 352 when the interface member 302 is in an unactuated position. Other suitable configurations of the protrusion and/or indentations of the interface assembly 301 are contemplated.

As depicted in FIG. 10A, when the interface member 302 is in the unactuated position, the valve stem 308 may be in the first configuration or second configuration. When the interface member 302 is the in unactuated position, a magnetic field between the first magnet 346 and the second magnet 348 may be relatively weak, but still bias the interface member 302 to the unactuated position. The interaction between the first protrusion 350 and the second protrusion may limit movement of the interface member 302 in a proximal direction relative to the collar member 304 in response to biasing of the magnetic fields of the first magnet 346 and the second magnet 348. FIG. 10B depicts the interface member 302 in the actuated position with the valve stem 308 in the third configuration. When the interface member 302 is in the actuated position, the magnetic field between the first magnet 346 and the second magnet 348 may be strong relative to when the interface member is in the unactuated position, so as to return the interface member 302 to the unactuated position when a force in the distal direction that is actuating the interface member to the actuated position is removed from the interface member 302.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1. An interface assembly for a valve of a medical device comprising:
a first portion;
a second portion; and
a biasing member integrally formed with the first portion and configured to bias the first portion in a proximal direction relative to the second portion; and
wherein the biasing member is configured to return the first portion to an unactuated position after the first portion is advanced in a distal direction relative to the second portion to an actuated position.

2. The interface assembly of claim 1, wherein the first portion, the second portion, and the biasing member are integrally formed from a monolithic single material.

3. The interface assembly of claim 1, wherein the biasing member and the first portion are formed from a monolithic single material and the biasing member is configured to engage the second portion as the first portion adjusts between the unactuated position and the actuated position.

4. The interface assembly of claim 1, wherein the biasing member comprises an ortho-planar spring extending between the first portion and the second portion.

5. The interface assembly of claim 4, wherein the ortho-planar spring comprises a plurality of legs extending between the first portion and the second portion, and the plurality of legs is circumferentially spaced about the first portion.

6. The interface assembly of claim 1, wherein the biasing member comprises a leaf-spring having two or more legs configured to engage the second portion.

7. The interface assembly of claim 6, wherein the biasing member comprises a plurality of leaf springs circumferentially spaced about the first portion and each leaf spring of the plurality of leaf springs is configured to engage the second portion.

8. The interface assembly of claim 1, wherein the biasing member comprises a first magnet at the first portion and a second magnet at the second potion.

9. The interface assembly of claim 8, wherein one or both of the first magnet and the second magnet is a ring magnet.

10. The interface assembly of claim 1, wherein the first portion comprises a through-hole.

11. The interface assembly of claim 1, wherein the first portion is configured to extend around an exterior of the second portion.

12. A valve assembly for a medical device, the valve assembly comprising;
a valve stem;
a valve interface assembly comprising:
an interface member coupled with the valve stem;
a collar member configured to receive the valve stem; and
a biasing member integrally formed with the interface member and including a plurality of legs extending to the collar member; and
wherein the biasing member is configured to return the valve stem to an unactuated position after the valve stem is advanced to an actuated position.

13. The valve assembly of claim 12, wherein a lumen through the valve stem is in fluid communication with a through-hole of the interface member.

14. The valve assembly of claim 12, wherein the interface member, the collar member, and the biasing member are integrally formed from a single unitary material.

15. The valve assembly of claim 12, wherein the biasing member and the interface member are formed from a monolithic single material and the biasing member is configured to engage the collar member as the interface member adjusts between the unactuated position and the actuated position.

16. The valve assembly of claim 12, wherein the interface member is configured to extend around an exterior of the collar member.

17. The valve assembly of claim 12, further comprising:
a valve body having a gas inlet passage and a gas outlet passage, and
wherein the valve stem is configured to translate within the valve body between the actuated position and the unactuated position.

18. A valve assembly for a medical device, the valve assembly comprising;
a valve stem;
a valve interface assembly comprising:
an interface member coupled with the valve stem;
a collar member configured to receive the valve stem; and
a biasing member comprising a first magnet integrally formed with the interface member and a second magnet at the collar member; and
wherein the biasing member is configured to return the valve stem to an unactuated position after the valve stem is advanced to an actuated position.

19. The valve assembly of claim 18, wherein the second magnet is integrally formed with the collar member.

20. The valve assembly of claim 18, wherein one or both of the first magnet and the second magnet are ring magnets extending circumferentially around the valve stem.
